(19) United States
(12) Patent Application Publication
(10) Pub. No.: US 2010/0175702 A1
(43) Pub. Date: Jul. 15, 2010

(54) PATIENT HARNESS SYSTEM FOR AN EMERGENCY COT

(76) Inventor: Scott Andrew West, Boondal (AU)

Correspondence Address: DINSMORE & SHOHL LLP, FIFTH THIRD CENTER, ONE SOUTH MAIN STREET, SUITE 1300, DAYTON, OH, 45402-2023, US

(21) Appl. No.: 12/377,133

(22) PCT Filed: Aug. 1, 2007
(86) PCT No.: PCT/US07/74951

§ 371 Date: Feb. 11, 2009

Related U.S. Application Data
(60) Provisional application No. 60/837,390, filed on Aug. 11, 2006.

(51) Int. Cl. A61F 5/37

(52) U.S. Cl. 128/876

(57) ABSTRACT

A patient harness system for an emergency cot comprising a chest strap restraint, a thigh strap restraint, and a harness restraint having a shoulder harness and pelvis restraint is disclosed. The harness restraint is mountable to the emergency cot via a plurality of quick release restraint buckles. A method of securing a patient to the emergency cot using the patient harness system is also disclosed.

The present invention relates generally to harnesses and in particular, to a patient harness system for an emergency cot.

Providing effective restraint for a patient in ambulances is a complex problem with many unique and unresolved issues. As the ambulance environment is specifically designed for emergency treatment of passengers, exposure in a crash environment may be more severe to a patient requiring transport. When transporting a patient with an acute medical problem that requires constant monitoring, a current practice is to restrain the patient directly to the cot with chest and hip belts. However, such a practice provides virtually no crash restraint in the forward direction.

It is against the above background that the present invention provides a patient harness system for an emergency cot which provides crash restraint, especially in the forward direction.

In one embodiment, a patient harness system for an emergency cot according to the present invention is disclosed. The patient harness system comprises a chest strap restraint; a thigh strap restraint; and a harness restraint having a shoulder harness and pelvis restraint. The harness restraint is mountable to the emergency cot via a plurality of quick release restraint buckles.

In another embodiment, a method of securing a patient to the emergency cot using the patient harness system according to the present invention is disclosed. The method comprises securing the chest strap restraint to the emergency cot; securing the thigh strap restraint to the emergency cot; and mounting the harness restraint to the emergency cot via the plurality of quick release restraint buckles.

These and other features and advantages of the invention will be more fully understood from the following description of the various embodiments of the invention taken together with the accompanying drawing. In particular, the organization and manner of operation of the invention, together with further objects and advantages thereof, may best be understood by reference to the following description taken in connection with the accompanying drawing in which:

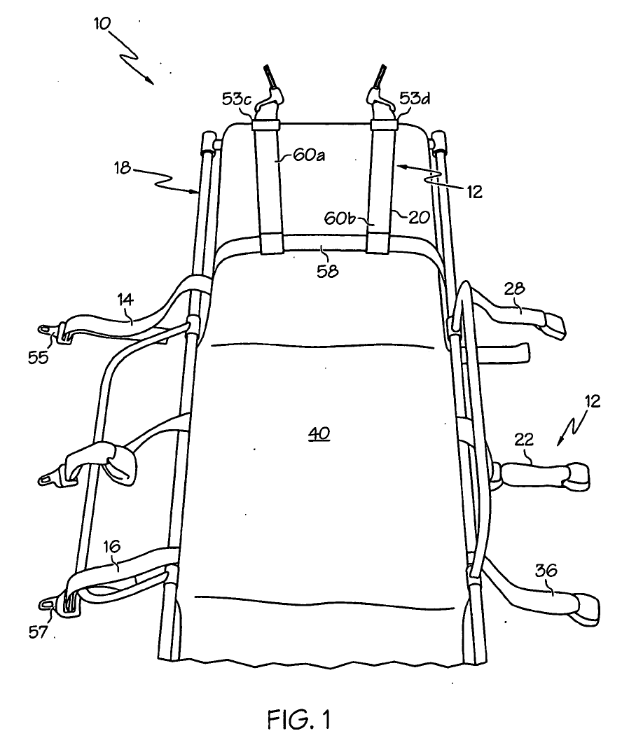

FIG. 1 is a front elevated perspective view of a patient restraint system according to the present invention, and shown provided to an emergency cot with a portion looping around a mattress with a fitted sheet thereof and prepared for use;

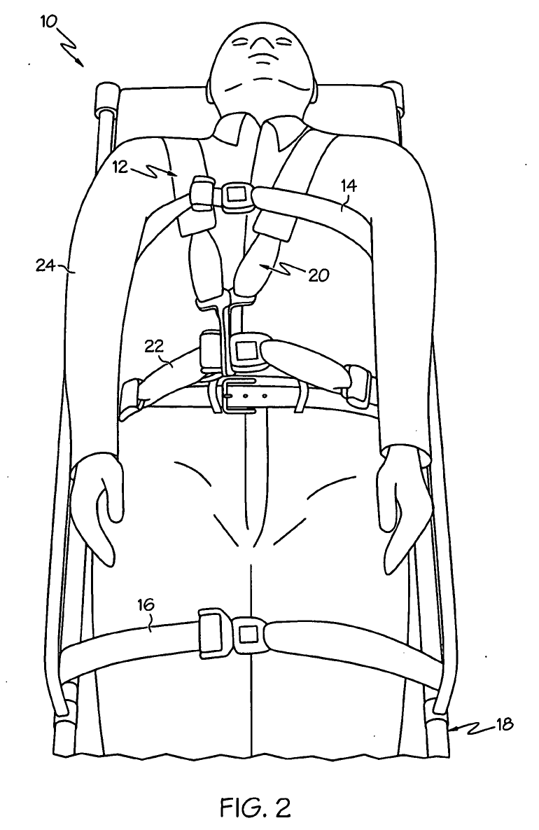

FIG. 2 is a front elevated perspective view of the patient restraint system of FIG. 1 securing a patient to an emergency cot;

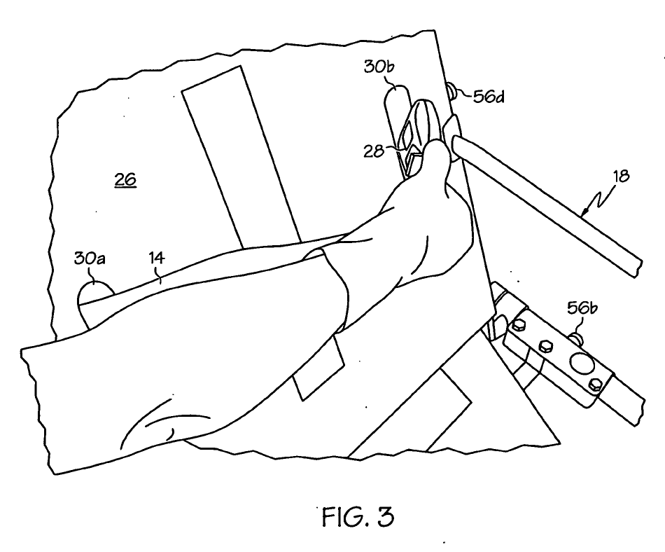

FIG. 3 is a side elevated perspective view showing the attaching of a chest strap restraint of the patient restraint system through a backrest panel of an emergency cot;

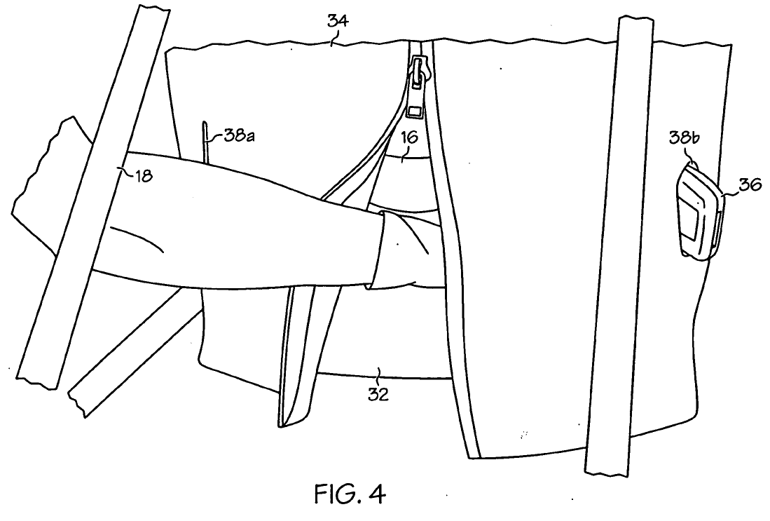

FIG. 4 is a bottom perspective view showing the threading of a thigh strap restraint of the patient restraint system through a seat panel of an emergency cot;

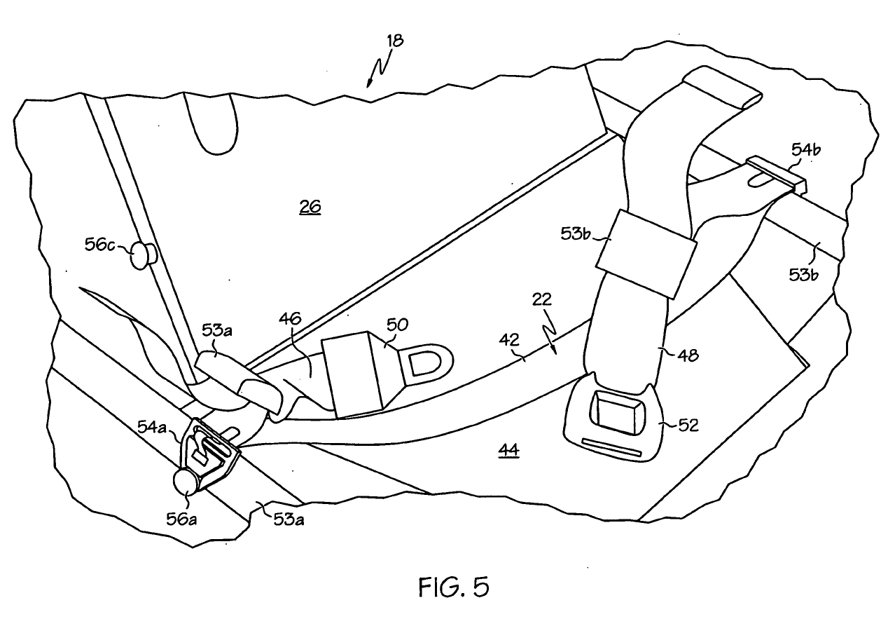

FIG. 5 is a side elevated, partial perspective view showing fitting of a pelvis strap of the patient restraint system on an emergency cot;

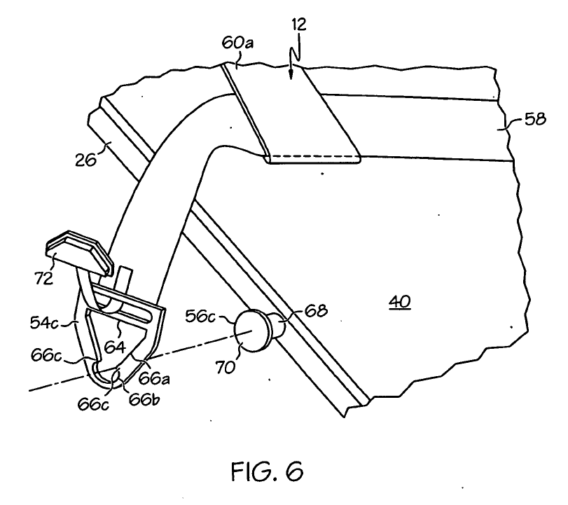

FIG. 6 is a side elevated, partial perspective view showing fitting of a quick release restraint buckle of the patient restraint system to an emergency cot;

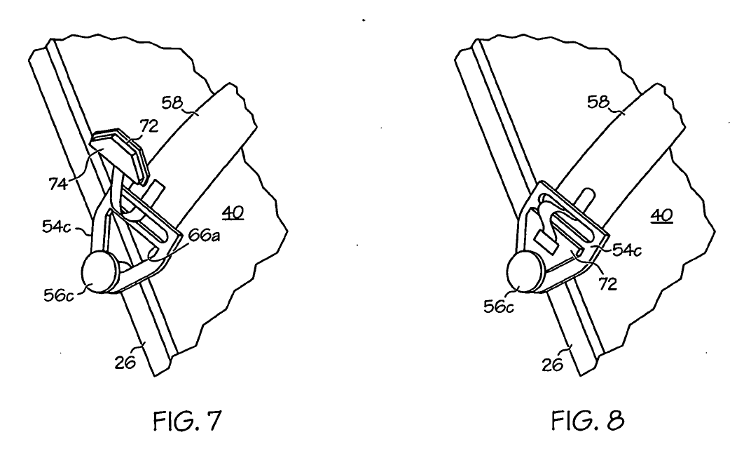

FIG. 7 is a side elevated, partial perspective view showing fitting of a retaining plug of the patient restraint system;

FIG. 8 is a side elevated, partial perspective view of the retaining plug of FIG. 7 properly fitted in place; and

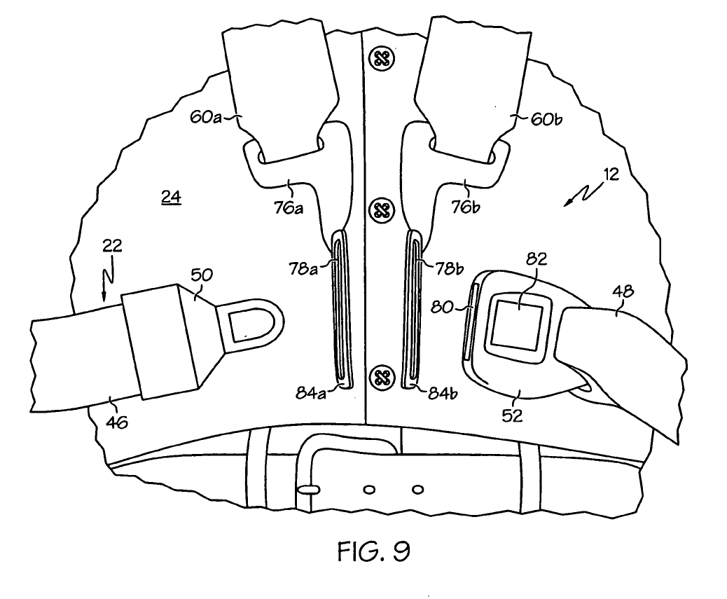

FIG. 9 is a front partial perspective view showing the feeding of a tang of a pelvis strap of the patient harness system through long slots of twisted tangs of a shoulder harness of the patent harness system according to the present invention.

While the invention may be susceptible to embodiments in different forms, there is shown in the drawing, and herein will be described in detail, specific embodiments with the understanding that the present disclosure is to be considered an exemplification of the principles of the invention, and is not intended to limit the invention to that as illustrated and described herein. Skilled artisans appreciate that elements in the figures are illustrated for simplicity and clarity and have not necessarily been drawn to scale. For example, the dimensions of some of the elements in the figures may be exaggerated relative to other elements, and with conventional parts removed, to help to improve understanding of embodiments of the present invention.

### Guidelines for Using Restraints

According to the present invention, in one illustrative embodiment, such as depicted by FIG. 1, disclosed is a patient harness system 10 having a harness restraint 12, a chest strap restraint 14, and a thigh strap restraint 16. The present invention provides the three restraints 12, 14, and 16 in order to secure a patient to an emergency cot 18 and to provide restraint of the patient in the forward direction during a crash. In one embodiment, the strap restraints 14 and 16 are two one-piece seven-foot restraints. The harness restraint 12 includes a shoulder harness 20 and a pelvis strap 22. A patient 24 restrained properly by the patient harness system 10 according to the present invention is shown by FIG. 2. A discussion on attaching the patient harness system 10 to the emergency cot 18 is provided hereafter with reference made to FIGS. 3-8.

### Attaching the Strap Restraints

The strap restraints 14 and 16 of the patient harness system 10 (FIG. 1) are attached to the emergency cot 18 by weaving the restraints through backrest and leg panels 26 and 32, respectively, of the emergency cot 18. As shown by FIG. 3, for example, with the chest strap restraint 14 unbuckled and the backrest panel 26 raised, a buckle 28 of the chest strap restraint 14 is passed from back to front through a first restraint slot 30a in the backrest panel 26. The buckle 28 is then pulled across the top of the backrest panel 26 and through the other restraint slot 30b. For the thigh strap restraint 16, for example, with the leg panel 32 of the cot 18 in the highest shock position and a fabric cover 34 normally provided thereon unzipped, a buckle 36 of thigh strap restraint 16 is treaded through a first cover slot 38a (partially shown) in the fabric cover 34, passed under the leg panel 32 of the cot 18 and through a second cover slot 38b on the other side of the fabric cover 34 as shown by FIG. 4. The fabric cover 34 is then zipped once the buckle 36 is through the second cover slot 38b. The strap restraints 14 and 16 are thus prepared for use as shown by FIG. 1.

### Attaching the Harness Restraint

As mentioned above, the harness restraint 12 has two parts—the shoulder harness 20 and the pelvis strap 22. Referring to FIG. 5, the pelvis strap 22 has a first pelvis strap segment 42 that spans above a seat panel 44 of the cot 18 such that the first pelvis strap segment 42 will be located between the seat panel 44 and a mattress 40 (FIG. 1) when the mattress is provided to the cot 18. Second and third pelvis strap segments 46 and 48 provide a tang 50 and a buckle 52, respectively, which when joined together are positioned on top of the patient 24 as shown by FIG. 2. Strap length adjusters 53a and 53b are also provided to the second and third pelvis strap segments 46 and 48, respectively. To lengthen a strap segment, for example, second pelvis strap segment 46, with the restraint strap 22 unbuckled, position the tang 50 and strap segment 46 perpendicular to the respective strap adjuster 53a and pull it away therefrom until the tang 50 is at the desired length. To shorten a strap segment, for example, second pelvis strap segment 46, position the loose end of the strap segment 46 perpendicular to the respective strap adjuster 53a and pull it away therefrom until the tang 50 is at the desired length. As the other strap length adjuster 53b, as well as the strap length adjuster 53c and 53d provided to the shoulder harness 20 as shown by FIG. 1, work in the same manner, for brevity, no further discussion regarding adjusters 53a, 53b, 53c, and 53d is provided.

Returning to FIG. 5, the pelvis strap 22 also provides a pair of quick release restraint buckles 54a and 54b, which are positioned between the pelvis strap segments 42 and 46, and 42 and 48, respectively. Each of the quick release restraint buckles 54a and 54b is mounted onto a mounting point 56a (same on side not shown by FIG. 5) provided on the cot 18. The quick release restraint buckles 54a and 54b are secured to the mounting points 56a (FIGS. 5) and 56b (FIG. 3) by pulling the restraint buckles upward to snap fit the restraint buckles in place. In one embodiment, the mounting points 56a and 56b for the pelvis strap 22 are provided on side longitudinally extending frame members 53a and 53b adjacent the seat panel 44. In other embodiments, the mounting points 56 may be located at any place on the cot 18 that is convenient in securing the quick release restraint buckles 54a and 54b to the cot 18 and still provide suitable use of the pelvis strap 22 as shown by FIG. 2. The snap fit employed by the quick release restraint buckles, according to the present invention, permits quick releasing of the buckles 54a and 54b without the use of a tool, and is further discussed hereafter in reference to attaching releasably, the shoulder harness 20 to the cot 18, and with reference made to FIGS. 6-8.

After fitting the mattress 40 (with or without linen) to the cot 18 and the second and third pelvis segments 46 and 48 of the pelvis strap 22 unbuckled as shown, for example, by FIG. 1, the shoulder harness 20 is then fitted by laying a securing strap segment 58 and shoulder strap segments 60a and 60b thereof on the mattress 40. As both sides of the securing strap segment 58 are the same, only one side is discussed and shown hereafter. The ends of the securing strap segment 58 each provide a quick release restraint buckle, such as quick release buckle 54c, which mounts to a respective mount point 56c provided on the lateral sides of the backrest panel 26 as shown by FIG. 6. The other mounting point 56d is shown by FIG. 3 to which the other quick release buckle (not shown) of the securing strap segment 58 of the harness restraint 12 attaches releasably.

As shown best by FIG. 6, the quick release buckle 54c provides a keyhole shaped slot 64 with an upper slot section 66a which tapers down to a lower slot section 66b. In between the upper and lower slot sections 66a and 66b is a detent section 66c which is slightly narrower than the diameter of a stem portion 68 of the mounting point 56c. Due to the narrower detent section 66c, positioning the stem portion 68 in the upper slot section 66a and pulling upwards on the quick release restraint buckle 56c will snap fit the stem portion 68 past the detent section 66c and be retained via an interference fit with the detent section 66c in the lower slot section 66b. A cap portion 70 of the quick release restraint buckle 56c, being wider than the lower slot section 66b, ensures that the quick release restraint buckle 56c cannot slip off the mounting point 56c as shown by FIG. 7.

Although the harness restraint 12, as shown and explained herein, is designed to be easily removed without the use of a tool to allow quick patient transfer, to avoid an unintentional release, the harness restraint 12 further provides a retaining plug 72 to each of the quick release restraint buckles. As shown by FIG. 7, the retaining plug 72 has a plug portion 74 which is shaped to fit tightly in the upper slot section 66a of the quick release restraint buckle 56c. With the plug portion 74 pushed into the upper slot section 66a, the retaining plug 72 prevents an unintentional downward pull on the restraint buckle 56c from quick releasing the stem portion 68 of the mounting point 56c from the lower slot section 66b as shown by FIG. 8. A discussion on securing the patient 24 to the emergency cot 18 with the patient harness system 10 is provided hereafter with reference made to FIGS. 1, 2, and 9.

Securing the Patient with the Restraints

To secure the patient 24 to the emergency cot 18 with the patient harness system 10, the shoulder strap segments 60a and 60b, and pelvis strap segments 46 and 48 are lengthened and the harness system unbuckled and arranged ready for fitment as shown by FIGS. 1 and 9. Once the patient 24 has been transferred to the cot 18 using approved EMS-procedures, the shoulder strap segments 60a and 60b are pulled over the shoulders of the patient and to the front of the patient as shown by FIG. 9. A pair of long twisted tangs 76a and 76b provided at the ends of the shoulder strap segments 60a and 60b are positioned adjacent each other. The pair of long twisted tangs 76a and 76b each provide a respective long slot 78a and 78b which are sized to permit the tang 50 of the pelvis strap 22 to fit therethrough and to be seated releasably in the engagement slot 80 of the buckle 52. A push button 82 on the buckle 52 is operated to release the tang 50 therefrom, which is the same for tangs 55 and 57 and buckles 28 and 36 provided on the ends of strap restraints 14 and 16, respectively (FIG. 1). After passing the tang 50 of the pelvis strap 22 through the long slots 80a and 80b and being seated securely in the engagement slot 80 of the buckle 52, opposed faces 84a and 84b of the twisted tangs will be in contact with each other and remain as such until as long as the pelvis strap 22 is buckled as shown by FIG. 2. Next, the shoulder strap segments 60a and 60b are adjusted until they are snug against the patients shoulders and the two long twisted tangs 76a and 76b are centrally positioned on patients torso as shown by FIG. 2 for adults (14 years and older or 46 KG or over) or centrally positioned on the boney area of the pelvis for children (3-14 years and 14-46 KG). Finally, the chest and thigh strap restraints 14 and 16 are fitted and secured over the chest and thigh of the patient 24 and adjusted as also shown by FIG. 2.

### Unfastening the Harness

The harness has been designed to make it easier to transfer patients. If the bedding is to be used in transferring the patient, the shoulder harness 20 should be removed but the other straps may be left attached to the cot 18. To do so, unfasten the chest, pelvis and thigh strap restraints 14, 12, and 16, respectively, and place the ends in a suitable position out of the way. Pull the shoulder strap segments 60a and 60b over the patient's head and position clear of the patient. Remove the retaining plug 72 on one of the backrest quick release buckles, for example, buckle 54c as shown by FIG. 7. Push down on the quick release buckle 54c and unclip it from the mounting point 56c as shown by FIG. 6. Pull the shoulder harness 20 up towards the patient's head and carefully slide the securing strap segment 58 out from under the patient 24 until the shoulder harness 20 is clear from the mattress 40. In this manner, the patient 24 positioned on the mattress 40 may be transferred from the cot 18.

The above-described embodiments are intended to illustrate the principles of the invention, not to limit its scope. Other embodiments, in variations to these preferred embodiments, will be apparent to those skilled in the art and may be made without departing from the spirit and scope of the invention as defined in the following claims.

1. A patient harness system for an emergency cot comprising:
a chest strap restraint;
a thigh strap restraint; and
a harness restraint having a shoulder harness and pelvis restraint, said harness restraint being mountable to the emergency cot via a plurality of quick release restraint buckles.

2. The patient harness system according to claim 1 wherein the shoulder harness includes a securing strap segment and a pair of shoulder strap segments.

3. The patient harness system according to claim 1 wherein the shoulder harness includes a securing strap segment and a pair of shoulder strap segments, and wherein the securing strap segment provides two of the plurality of quick release restraint buckles.

4. The patient harness system according to claim 1 wherein the pelvis strap provides two of the plurality of quick release restraint buckles.

5. The patient harness system according to claim 1 wherein the shoulder harness includes a securing strap segment and a pair of shoulder strap segments, wherein the plurality of quick release restraint buckles is four quick release restraint buckles, and wherein the securing strap segment provides two of the four quick release restraint buckles and the pelvis strap provides the other two of the four quick release restraint buckles.

6. The patient harness system according to claim 1 wherein the shoulder harness includes a securing strap segment and a pair of shoulder strap segments, wherein the emergency cot provides mounting points for the plurality of quick release restraint buckles such that the securing strap segment and the pelvis strap mount releasably to the mounting points via the quick release restraint buckles.

7. The patient harness system of claim 1 mounted to the emergency cot, wherein the shoulder harness includes a securing strap segment and a pair of shoulder strap segments, wherein the emergency cot provides mounting points on a backrest panel thereof, and wherein said securing strap segment is mounted releasably to the mounting points via a pair of the plurality of quick release restraint buckles.

8. The patient harness system according to claim 2 wherein the pair of shoulder strap segments provides a pair of long twisted tangs.

9. The patient harness system according to claim 2 wherein the pelvis strap and the securing strap segments mount releasably to the emergency cot via the plurality of quick release restrain buckles, and wherein the pair of shoulder strap segments provides a pair of long twisted tangs.

10. The patient harness system according to claim 2 wherein the pelvis strap and the securing strap segment mounts releasably to the emergency cot via the plurality of quick release restraint buckles, wherein the pair of shoulder strap segments provides a pair of twisted tangs each with a slot, and wherein the pelvis strap provides a buckle and a tang, the tang being sized to fit through each slot of the twisted tangs when secured to the buckle.

11. The patient harness system according to claim 1 further comprising a retaining plug for each of the plurality of quick release restraint buckles.

12. The patient harness system according to claim 1 further comprising a retaining plug for each of the plurality of quick release restraint buckles, wherein each of the plurality of quick release restraint buckles has an upper slot section, and the retaining plug has a plug portion sized to fit securely in the upper slot section.

13. The patient harness system according to claim 1 further comprising a retaining plug for each of the plurality of quick release restraint buckles, wherein each of the plurality of quick release restraint buckles has an upper slot section, a lower slot section, and a detent section, and the retaining plug has a plug portion sized to fit securely in the upper slot section.

14. The patient harness system according to claim 1 wherein each of the plurality of quick release restraint buckles has an upper slot section, a lower slot section, and a detent section sized to snap fit a mounting point provided to the emergency cot from the upper slot section to the lower slot section.

15. The patient harness system according to claim 1 wherein each of the plurality of quick release restraint buckles has an upper slot section, a lower slot section, and a detent section sized to snap fit a mounting point provided to the emergency cot from the upper slot section to the lower slot section, and said patient harness system further comprising a retaining plug for each of the plurality of quick release restraint buckles, the retaining plug has a plug portion sized to fit securely in the upper slot section.

16. The patient harness system according to claim 1 wherein the shoulder harness includes a securing strap segment and a pair of shoulder strap segments, wherein the pelvis strap and the securing strap segment mounts releasably to the emergency cot via the plurality of quick release restrain buckles, wherein the pair of shoulder strap segments provides a pair of twisted tangs each with a slot, wherein the pelvis strap provides a buckle and a tang, the tang being sized to fit through each slot of the twisted tangs when secured to the buckle, wherein each of the plurality of quick release restraint buckles has an upper slot section, a lower slot section, and a detent section sized to snap fit a mounting point provided to the emergency cot from the upper slot section to the lower slot section, and wherein said patient harness system further comprising a retaining plug for each of the plurality of quick release restraint buckles, the retaining plug has a plug portion sized to fit securely in the upper slot section.

17. A method of securing a patient to the emergency cot using the patient harness system according to claim 1, said method comprising:
securing the chest strap restraint to the emergency cot;
securing the thigh strap restraint to the emergency cot; and
mounting the harness restraint to the emergency cot via the plurality of quick release restraint buckles.

18. The method according to claim 17 wherein mounting the harness restraint includes snap fitting the plurality of quick release restraint buckles to mounting points provided on a backrest panel and longitudinally extending frame members thereof, and said method further comprises installing a retaining plug in each of the plurality of quick release restraint buckles.

19. The method according to claim 18 wherein the shoulder restraint includes a pair of shoulder strap segments providing a pair of twisted tangs each with a slot, and wherein the pelvis strap provides a buckle and a tang, the tang being sized to fit through each slot of the twisted tangs when secured to the buckle, said method further comprising placing the patient on the emergency cot, placing the shoulder strap segments over the patient, and securing to the buckle the tang fitted through each slot of the twisted tangs.

20. The method according to claim 19 further comprising further securing the patient to the emergency cot via the chest and thigh strap restraints.
